(19) United States
(12) Patent Application Publication
(10) Pub. No.: US 2009/0111774 A1
(43) Pub. Date: Apr. 30, 2009

(54) PMEA LIPID CONJUGATES

(75) Inventors: Marc Tokars, Douglassville, PA (US); Richard Lawrence, Calverton, NY (US); Matthews Bradley, Annapolis, MD (US)

Correspondence Address: WOLF GREENFIELD & SACKS, P.C., 600 ATLANTIC AVENUE, BOSTON, MA, 02210-2206, US

(73) Assignee: Luitpold Pharmaceuticals, Inc., Shirley, NY (US)

(21) Appl. No.: 12/130,911

(22) Filed: May 30, 2008

Related U.S. Application Data
(60) Provisional application No. 60/941,403, filed on Jun. 1, 2007.

(51) Int. Cl. A61K 31/675; C07F 9/547; A61P 31/12

(52) U.S. Cl. 514/81; 544/244

(57) ABSTRACT

The present invention relates to PMEA lipid conjugates and to methods of using the conjugates to treat diseases caused by viruses such as herpes, cytomegalovirus, varicella, paramyxovirus, polyoma virus, and human papillomavirus. Methods for making the PMEA lipid conjugates are also provided.

### RELATED APPLICATION

This application claims the benefit under 35 U.S.C. §119(e) of United State provisional application Ser. No. 60/941,403, filed Jun. 1, 2007, the entire contents of which are incorporated by reference herein.

### FIELD OF THE INVENTION

The present invention relates to PMEA lipid conjugates and to methods of using the conjugates to treat viral diseases.

### BACKGROUND OF THE INVENTION

Several hundred different types of viruses are known to cause disease. Of these viruses, many infect their hosts without producing overt symptoms, while others (e.g., influenza) produce a well-characterized set of symptoms. Symptoms can vary with the virulence of the infecting strain and identical viral strains can have drastically different effects depending upon the health and immune response of the host.

Despite advances in treatment and in the development of vaccines for certain viral diseases (e.g., polio and measles), and the eradication of specific viruses from the human population (e.g., smallpox), viral diseases remain an important medical and public health problem and have a debilitating effect on the economic output of society. The diagnosis of viral diseases is frequently difficult and the availability of treatments is limited.

A number of antiviral agents that interfere with viral replication have been developed. Most of the antiviral agents currently available interfere with the nucleic acid synthesis of the viruses. Nucleotide or nucleoside analogues are such a group of antiviral agents. Examples of nucleotide or nucleoside analogues include azidothymidine (AZT), dideoxyinosine (ddI), dideoxycytosine (ddC), stavudine (D4T), and 9-(2-phosphonylmethoxyethyl)adenine (PMEA).

PMEA, 9-(2-phosphonylmethoxyethyl)adenine, is an acyclic nucleoside phosphonate. It is considered to be among the most active agents tested against both hepatitis B and human immunodeficiency virus (HIV). PMEA is commonly called adefovir and the terms “PMEA” and “adefovir” are used interchangeably. PMEA failed in clinical development due to nephrotoxicity and poor oral availability.

Because viruses are small and replicate inside cells using the cell's molecular machinery, there are only a limited number of metabolic pathways or functions that antiviral agents can target. Moreover, antiviral agents may be toxic to host cells and viruses can develop resistance to antiviral agents. Therefore, it is difficult to develop antiviral agents.

One area of antiviral chemotherapy research focusses on improving the pharmacokinetics and the selectivity of existing antiviral agents. Extensive research has been done on the use of lipid molecules, such as fatty acids, to improve selectivity of drugs for their target tissues. Fatty acids have been conjugated to drugs to help the drugs cross the blood brain barrier. For example, docosahexaenoic acid (DHA), a 22 carbon naturally-occurring unbranched fatty acid, previously has been shown to be unusually effective in allowing a drug conjugated to it cross the blood brain barrier.

Examples of the conjugation of fatty acids to nucleoside or nucleoside analogues are described in U.S. Pat. Nos. 5,216,142 and 5,276,020. U.S. Pat. Nos. 5,216,142 and 5,276,020 describe methods of enhancing transport of antivirals across lipid barriers in the body and methods of treating viral infections using nucleoside or nucleoside fatty acid analogues.

Other examples of the conjugation of lipid molecules such as fatty acids, fatty alcohols and fatty amines to pharmaceutical agents are described in U.S. Pat. Nos. 5,919,815, 5,795,909, 5,580,899, and US patent applications 2003/0065023 and 2002/0177609. The benefits of pharmaceutical agent-lipid conjugates described in the aforementioned patent documents include: targeting of the pharmaceutical agent to the tissue of interest, favorably affecting the volume of distribution of the pharmaceutical agent in the tissue of interest, and reducing toxicity and side effects of the drug. Another described benefit of the pharmaceutical agent-lipid conjugates is that once the lipid is separated from conjugation to the pharmaceutical agent(s) in vivo, the lipid can be readily metabolized in the body.

The type of lipid molecules employed have included phospholipids, non-naturally occurring branched and unbranched fatty acids, and naturally occurring branched and unbranched fatty acids, fatty alcohols and fatty amines ranging from as few as 4 carbon atoms to more than 30 carbon atoms. In one instance, enhanced receptor binding activity was observed (for an adenosine receptor agonist), and it was postulated that the pendant lipid molecule interacted with the phospholipid membrane to act as a distal anchor for the receptor ligand in the membrane micro environment of the receptor. This increase in potency, however, was not observed when the same lipid derivatives of adenosine receptor antagonists were used, and generalizations thus were not made possible by those studies.

The exact mechanism by which lipid molecules such as fatty acids help agents conjugated to them cross the blood brain barrier is not yet fully understood. It is believed that the attachment of the lipid molecules to hydrophilic agents renders these agents more hydrophobic (more lipophilic) than unconjugated agents. This increased lipophilicity is believed to help the agents cross the blood brain barrier. Increased lipophilicity has also been suggested as a mechanism for enhancing intestinal uptake of agents into the lymphatic system, thereby enhancing the entry of the conjugate into the brain and also thereby avoiding first-pass metabolism of the conjugate in the liver. Once at or near the tissue target, some have reported, supported by data, that the lipid molecule-agent conjugate must be converted back to the parent agent to become effective.

### SUMMARY OF THE INVENTION

The present invention involves the unexpected finding that particular PMEA lipid conjugates, a PMEA-oleyl amide (compound of Formula III) and a PMEA-oleyl carbamate (compound of Formula VI), were active against particular viruses whereas unconjugated PMEA was inactive against the same types of viruses. These results are unexpected because previous lipid molecules-agent conjugates, such as DHA-paclitaxel, were inactive as intact conjugates and only had therapeutic activity after metabolism back to the parent agent (Bradley et al., Clin. Cancer Res. 7(10): 3229-3238, 2001). These findings indicate that the PMEA conjugates either themselves possess, or cause to form, antiviral activity beyond that of the parent agent (PMEA) itself.

The invention also involves the unexpected finding that among the viruses that are sensitive to compound of Formula III or compound of Formula VI but not to PMEA, some were sensitive to both compound of Formula III and compound of Formula VI while others were only sensitive to either compound of Formula III or to compound of Formula VI but not the other.

Compounds of Formula III and Formula VI were studied for antiviral activity on twenty four types of viruses. Two types of viruses, varicella-zoster virus (VZV) and polyoma (BK) virus, were sensitive to the compound of Formula III and the compound Formula VI. Four types of viruses, herpes simplex virus type 1 (HSV-1), herpes simplex virus type 2 (HSV-2), measles virus, and human papillomavirus (HPV) were sensitive only to the compound of Formula III and one type of virus, herpes cytomegalovirus (HCMV), was sensitive only to the compound of Formula VI. The other tested types of viruses showed very little or no sensitivity to either compound of Formula III or compound of Formula VI.

There was no correlation between the type of PMEA lipid linkage, oleyl amide (as in compound of Formula I) or oleyl carbamate (as in compound of Formula IV), and the sensitivity of the virus. These findings are further illustrated in the Examples below. Based on the teachings of the prior art, one of ordinary skill in the art would not be able to predict the sensitivity of the viruses to compound of Formula III or compound of Formula VI because, like in this instance, there are no general rules that predict which lipid or which type of lipid conjugation improves the antiviral activity of PMEA.

According to one aspect of the invention, a compound having a structure:

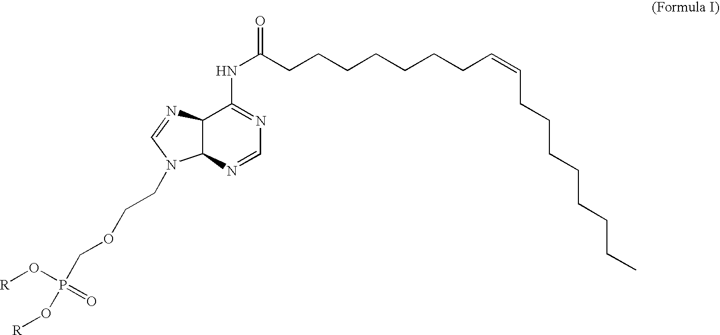

or a salt thereof is provided, wherein R is —CH₂CH₃, hydrogen or a monovalent cation. The monovalent cation may be lithium, sodium or potassium.

In one embodiment, the compound is:

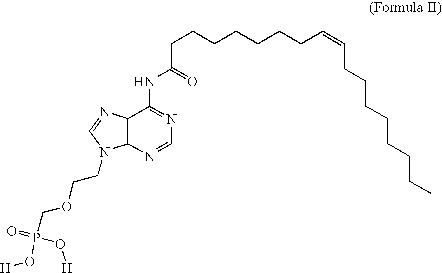

In one embodiment, the compound is:

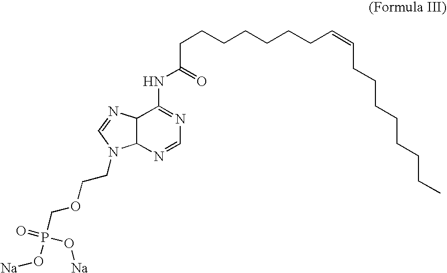

According to another aspect of the invention, a compound having a structure:

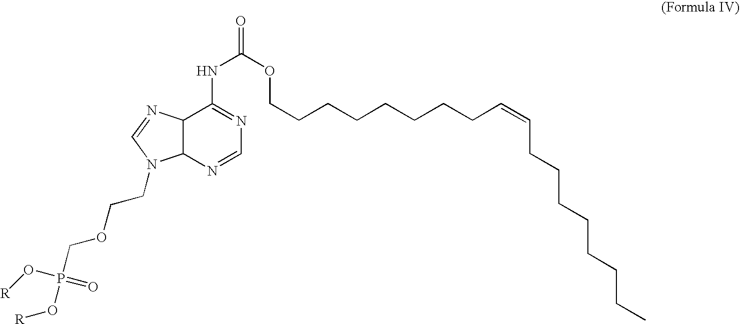

or a salt thereof is provided, wherein R is —CH₂CH₃, hydrogen or a monovalent cation. The monovalent cation may be lithium, sodium or potassium.

In one embodiment, the compound is:

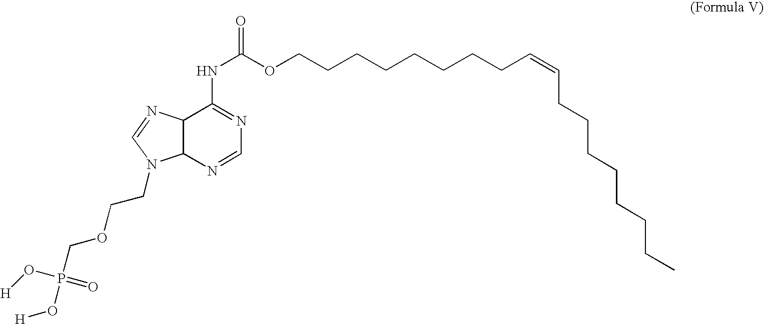

In one embodiment, the compound is:

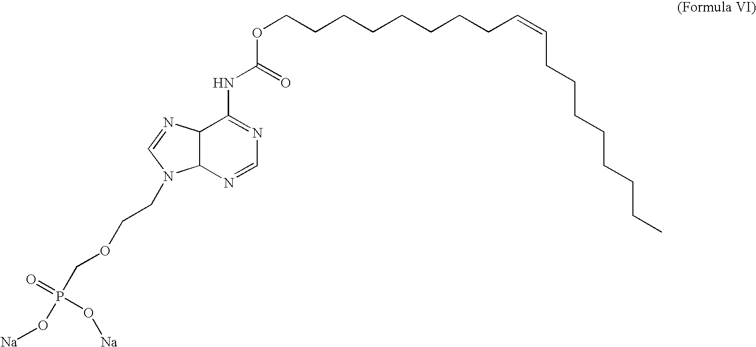

According to another aspect of the invention, a pharmaceutical composition is provided. The pharmaceutical composition comprises the compound of Formula I or the compound of Formula IV and a pharmaceutically acceptable carrier. The pharmaceutical composition may further comprise an agent other than the compound of Formula I and/or the compound of Formula IV.

In some embodiments, the agent is an antiviral agent. Examples of antiviral agents include, but are not limited to, acyclovir, cidofovir famciclovir, foscarnet, gancyclovir, penciclovir, trifluridine, valacyclovir, valganciclovir, and vidarabine.

In one embodiment, a method for treating a subject having a viral disease is provided. The method comprises administering to the subject an effective amount of a pharmaceutical composition of the compound of Formula I to treat the viral disease wherein the viral disease is caused by herpes simplex virus (HSV), varicella-zoster virus (VZV), measles virus, polyoma (BK) virus, or human papillomavirus (HPV).

In one embodiment, a method for treating a subject having a viral disease is provided. The method comprises administering to the subject an effective amount of a pharmaceutical composition of the compound of Formula IV to treat the viral disease wherein the viral disease is caused by cytomegalovirus (CMV or HCMV), varicella-zoster virus (VZV), or polyoma (BK) virus.

The herpes simplex virus may be herpes simplex type I or herpes simplex type 2 virus. The herpes simplex virus type I disease may be oral herpes infection. The herpes simplex virus type II disease may be genital herpes infection. The varicella-zoster virus disease may be chickenpox or shingles.

These and other aspects of the invention, as well as various advantages and utilities, will be more apparent with reference to the detailed description of the invention. Each aspect of the invention can encompass various embodiments, as will be understood.

### DETAILED DESCRIPTION OF THE INVENTION

The invention described herein relates to PMEA lipid conjugates and methods of using the conjugates in the treatment of viral diseases. The invention provides compositions of matter.

The invention also encompasses methods of preparing and conjugating PMEA to fatty acids (e.g., oleic acid) and fatty alcohols (e.g., oleyl alcohol). Examples of methods and processes of making the compositions are described herein, although one of ordinary skill in the art will recognize that there may be other possible synthetic methods.

PMEA is an acyclic nucleoside phosphonate. PMEA is 9-(2-phosphonylmethoxyethyl) adenine and has the structure:

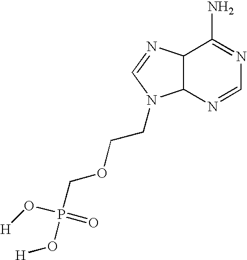

The accepted mechanism of action of PMEA is as an alternate substrate for dATP. The product, PMEApp, acts as a potent DNA chain terminator, which explains its antiviral activity. Uptake of PMEA by human cells and subsequent conversion to the mono- and diphosphorylated metabolites (PMEAp and PMEApp) are dose-dependent and occur proportionally with the initial extracellular PMEA concentrations. Adenylate kinase is unable to phosphorylate PMEA. However, 5-phosphoribosyl-1-pyrophosphate synthetase directly converts PMEA to PMEApp with a Km of 1.5 mM and a Vmax that is 150-fold lower than the Vmax for AMP. ATPase, 5′-phosphodiesterase, and nucleoside diphosphate kinase are able to dephosphorylate PMEApp to PMEAp, however, to a much lower extent than the dephosphorylation of ATP. PMEApp has a relatively long intracellular half-life (16-18 hours) and has a much higher affinity for the human immunodeficiency virus-specified reverse transcriptase and other viral transcriptases (hepadnavirus and others) than for the cellular DNA polymerase alpha (Ki/Km: 0.01 and 0.60, respectively). PMEApp is at least as potent an inhibitor of human immunodeficiency virus reverse transcriptase as 2′,3′-dideoxyadenosine 5′-triphosphate. Being an alternative substrate to dATP, PMEApp acts as a potent DNA chain terminator, and this may explain its anti-retrovirus and other antiviral activity.

Oleic acid is a monounsaturated omega-9 fatty acid found in various animal and vegetable sources. It has the formula C₁₈H₃₄O₂ (or CH₃(CH₂)₇CH═CH(CH₂)₇COOH). Oleic acid is found in various animal and vegetable sources and can be isolated form these natural sources or it can be chemically synthesized.

Oleyl alcohol, octadecenol, or cis-9-octadecen-1-ol, is a fatty alcohol. Its chemical formula is C₁₈H₃₆O or CH₃(CH₂)₇—CH═CH—(CH₂)₈OH. Oleyl alcohol is found in various animal and vegetable sources. It can be isolated form these natural sources or it can be chemically synthesized from oleic acid, natural or synthetic. Oleyl alcohol may be made by converting oleic acid to oleyl alcohol using standard methods. For example, oleyl alcohol can be synthesized from oleic acid by forming oleic acid methyl ester and then reducing the ester with, for example, NaBH₄, LiAlH₄ or another reducing agent.

The invention provides compositions of matter. In one aspect of the invention, the composition of matter is compound of Formula I. The invention also provides for a composition of matter of compound of Formula IV.

The invention also provides pharmaceutical compositions comprising compound of Formula I or compound of Formula IV. The pharmaceutical composition comprises the compound of Formula I or the compound of Formula IV in a pharmaceutically acceptable carrier or diluent.

The term “pharmaceutically acceptable carrier” as used herein refers to compounds suitable for use in contact with recipient subjects, preferably mammals, and more preferably humans, and having a toxicity, irritation, or allergic response commensurate with a reasonable benefit/risk ratio, and effective for their intended use. In some embodiments, the pharmaceutically acceptable carrier is an aqueous solution (e.g., saline).

The pharmaceutical compositions also can contain other components useful in formulating pharmaceutical preparations for administration to subjects, preferably humans, including surfactants, solvents, preservatives, diluents, buffering agents and the like, all of which are standard in the pharmaceutical arts.

Suitable surfactants for use with the present invention include nonionic agents, such as long-chain fatty acids and their water-insoluble derivatives. These include fatty amines such as lauryl acetyl and stearyl amine, glyceryl esters such as the naturally occurring mono-, di- and triglycerides, and fatty acid esters of fatty amines, such as propylene glycol, polyethylene glycol, sorbitan, sucrose and cholesterol. Also useful are compounds that have polyoxyethylene groups added through an ether linkage with an amine group. Compounds that are also useful in the present invention include the polyoxyethylene sorbitan fatty acid esters and polyoxyethylene glycerol and steroidal esters. Some of the preferred surfactants are Cremophor® EL and Cremophor® EL-P, which are polyoxyethylated castor oil surfactants.

It is contemplated that other surfactants may be used to solubilize the compositions described herein. For example, it is contemplated that polysorbate 80, polysorbate 20, sodium laurate, sodium oleate, and sorbitan monooleate may be useful in certain embodiments of the present invention. Anionic surfactants may also be useful in the practice of the present invention. Examples of these include, but are not limited to, sodium cholate, sodium lauryl sulfate, sodium deoxycholate, sodium laurate, sodium oleate, and potassium laurate.

In certain embodiments, dehydrated ethanol may be used as a solvent for the compositions described herein. In other embodiments, glycols such as propylene glycol or polyethylene glycol are within the scope of the invention. Simple complex polyols may also be suitable solvents. Moreover, the use of non-dehydrated amines may also be suitable within the scope of the present invention. It is recognized that the determination of a solvent and its proper concentration to fully solubilize the conjugate, such as compound of Formula III or compound of Formula VI compositions is within the scope of a skilled artisan, and would not require undue experimentation.

Suitable buffering agents include: acetic acid and a salt (1-2% W/V); citric acid and a salt (1-3% W/V); and phosphoric acid and a salt (0.8-2% W/V).

Suitable preservatives include antimicrobial agents, such as, benzalkonium chloride (0.003-0.03% W/V); chlorobutanol (0.3-0.9% W/V); parabens (0.01-0.25% W/V) and thimerosal (0.004-0.02% W/V) and/or suitable antiantioxidants, such as, ascorbic acid, ascorbyl pamitate, BHA, BHT, hypophosphorous acid, monothioglycerol, potassium metabisulfite, propyl gallate, sodium formaldehyde sulfoxylate, sodium metabisulfite, sodium bisulfite, sodium thiosulfate, sulfur dioxide, tocopherol and/or tocopherols excipient.

In some embodiments, the compounds of the invention (e.g., compounds of Formula I, II, III, IV, V, or VI) are provided in the form of a pharmaceutically acceptable salt. By “pharmaceutically acceptable salt” is meant those salts which are, within the scope of sound medical judgment, suitable for use in contact with the tissues of a subject without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 66:1 (1977). The salts may be prepared during the final isolation and purification of the compounds of the invention or separately. The salts may be prepared by reacting a free base function with a suitable acid to form the salt (acid addition salts) or by reacting a carboxylic acid-containing moiety with a suitable base (base addition salts). Examples of suitable bases include hydroxide, carbonate, or bicarbonate of a pharmaceutically acceptable metal cation or with ammonia or organic primary, secondary, or tertiary amine.

Representative acid addition salts include, but are not limited to acetate, adipate, alginate, citrate, aspartate, benzoate, benzenesulfonate, bisulfate, butyrate, camphorate, camphorolsulfonate, digluconate, glycerophosphate, hemisulfate, heptanoate, hexanoate, fumarate, hydrochloride, hydrobromide, hydroiodide, 2-hydroxyethanesulfonate (isothionate), lactate, maleate, methanesulfonate, nicotinate, 2-Naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate. Examples of acids that can be employed to form pharmaceutically acceptable acid addition salts include inorganic acids, such as hydrochloric acid, hydrobromic acid, sulfuric acid, and phosphoric acid, and organic acids, such as oxalic acid, maleic acid, succinic acid, and citric acid.

Representative pharmaceutically acceptable basic addition salts include, but are not limited to, cations based on alkali metals or alkaline earth metals, such as lithium, sodium, potassium, calcium, magnesium, and aluminum, and the like, and nontoxic quaternary ammonia and amine cations including ammonium, tetramethylammonium, tetraethyl-ammonium, methylamine, dimethylamine, trimethylamine ethylamine, diethylamine, triethylamine, and the like. Other representative organic amines useful for the formation of base addition salts include ethylenediamine, ethanolamine, diethanolamine, piperidine, piperazine, and the like.

In one aspect, the pharmaceutical compositions comprise a compound of Formula I, a compound of Formula II, a compound of Formula III, a compound of Formula IV, a compound of Formula V, or compound of Formula VI and one or more therapeutic agents. In some preferred embodiments, the therapeutic agent is one or more antiviral agent(s). Examples of antiviral agents that may be used in the invention include, but are not limited to: acyclovir, cidofovir, famciclovir, foscarnet, gancyclovir, penciclovir, trifluridine, valacyclovir, valganciclovir, and vidarabine. Other examples of antiviral agents that may be used in the invention include Acemannan, Acyclovir Sodium, Alovudine, Alvircept Sudotox, Amantadine Hydrochloride, Aranotin, Arildone, Atevirdine Mesylate, Avridine, Azidothymidine (AZT), BRL 47923, BRL 44385, Cipamfylline, Cytarabine Hydrochloride, Delavirdine Mesylate, Desciclovir, Didanosine, Dideoxycytosine (ddC), Dideoxyinosine (ddI0, Disoxaril, Edoxudine, Enviradene, Enviroxime, Famotine Hydrochloride, Fiacitabine, Fialuridine, Fosarilate, Foscamet Sodium, Fosfonet Sodium, Gancyclovir Sodium, Idoxuridine, Indinavir, Kethoxal, Lamivudine, Lobucavir, Memotine Hydrochloride, Methisazone, Nelfinavir, Nevirapine, Pirodavir, Ribavirin, Rimantadine Hydrochloride, Ritonavir, Saquinavir Mesylate, Somantadine Hydrochloride, Sorivudine, Statolon, Stavudine, Tilorone Hydrochloride, Valacyclovir Hydrochloride, Vidarabine, Vidarabine Phosphate, Vidarabine Sodium Phosphate, Viroxime, Zalcitabine, Zidovudine, Zinviroxime, integrase inhibitors, TMC-125 and TMC-114. Antivirals also include nucleoside analogs, nonnucleoside reverse transcriptase inhibitors, protease inhibitors, and integrase inhibitors.

The present invention also provides methods of treating a viral disease in a subject, comprising administering to the subject an effective amount of a pharmaceutical compound comprising a compound of Formula I, a compound of Formula II, a compound of Formula III, a compound of Formula IV, a compound of Formula V, or a compound of Formula VI. Preferably, the methods are employed to inhibit certain viral diseases in a subject, such as a mammal. Methods of the invention also are readily adaptable for use in assay systems, e.g., assaying viral replication and proliferation and properties thereof, as well as identifying compounds that affect viral disease.

As used herein, a subject includes a mammal, such as a human, non-human primate, cow, rabbit, horse, pig, sheep, goat, dog, cat, or rodent such a rat, mouse or a rabbit. In some embodiments, the subject is a human.

The products and methods of the invention are useful for treating certain viral diseases. Viral diseases treatable by compounds of the invention include, for example, diseases caused by varicella-zoster virus (VZV), polyoma (BK) virus, herpes simplex virus type 1 (HSV-1), herpes simplex virus type 2 (HSV-2), measles virus and herpes cytomegalovirus (HCMV).

Varicella-zoster virus (VZV) causes chickenpox. The disease is a highly contagious and is spread by airborne droplets of moisture containing the varicella-zoster virus. Before the introduction of a vaccine in 1995, about 90% of children developed chickenpox by age 15. A subject with chickenpox is most contagious just after symptoms start but remains contagious until the last blisters have crusted.

Although most subjects with chickenpox simply have sores on the skin and in the mouth, the virus sometimes infects the lungs, brain, heart, or joints. Such serious infections are more common in newborns, adults, and subjects with an impaired immune system.

A subject who has had chickenpox develops immunity and cannot contract it again. However, the varicella-zoster virus remains dormant in the body after an initial infection with chickenpox, sometimes reactivating in later life, causing shingles.

The symptoms of chickenpox begin 10 to 21 days after infection. They include mild headache, moderate fever, loss of appetite, and a general feeling of illness (malaise). Younger children often do not have these symptoms, but symptoms are often severe in adults. About 24 to 36 hours after the first symptoms begin, a rash of small, flat, red spots appears. The spots usually begin on the trunk and face, later appearing on the arms and legs. Over 6 to 8 hours, each spot becomes raised; forms an itchy, round, fluid-filled blister against a red background; and finally crusts. Spots continue to develop and crust for several days. The spots may become infected by bacteria, causing erysipelas, pyoderma, cellulitis, or bullous impetigo. New spots usually stop appearing by the fifth day, the majority are crusted by the sixth day, and most disappear in fewer than 20 days.

Spots in the mouth quickly rupture and form raw sores (ulcers), which often make swallowing painful. Raw sores may also occur on the eyelids and in the upper airways, rectum, and vagina. Spots in the larynx and upper airways may occasionally cause severe difficulty in breathing. Lymph nodes at the side of the neck may become enlarged and tender.

Lung infection occurs in about 1 out of 400 subjects with chickenpox, especially adolescents and adults, resulting in cough and difficulty breathing. Brain infection (encephalitis) is less common and produces unsteadiness in walking, headache, dizziness, confusion, and seizures. Heart infection sometimes causes a heart murmur. Joint inflammation produces joint pain.

The diagnosis of chickenpox is usually made based on clinical presentation because the rash and the other symptoms are so typical. Measurement of the levels of antibodies in the blood and laboratory identification of the virus may be performed but are not usually needed.

Shingles and chickenpox are both caused by the varicella-zoster virus. Chickenpox is the initial infection with varicella-zoster virus, and shingles is a re-emergence of the virus, usually years later. During the chickenpox infection, the virus spreads in the bloodstream and infects many nerve cells (ganglia) of the spinal or cranial nerves, remaining there in a dormant (latent) state. The virus may never cause symptoms again, or it may reactivate many years later. When it reactivates, the virus travels back down the nerve fibers to the skin, where it creates painful sores resembling those of chickenpox. This outbreak of sores (shingles) is almost always limited to a strip of the skin on one side of the body that contains a group of infected nerve fibers, called a dermatome.

Shingles may develop at any age but is most common after age 50. Most often, the reason for reactivation is unknown, although reactivation sometimes occurs when the body's immunity is reduced by another disorder, such as AIDS or Hodgkin's disease, or by use of drugs that impair the immune system.

Some subjects with shingles feel unwell and have chills, a fever, nausea, diarrhea, or difficulties with urination in the 3 or 4 days before shingles develops. Others experience pain, a tingling sensation, or itching in an area of skin. Clusters of small, fluid-filled blisters surrounded by a small red area then develop. The blisters occupy only the limited area of skin served by the infected nerves. Most often, blisters appear on the trunk, usually on only one side. However, a few blisters may appear elsewhere as well. The involved area of the body is usually sensitive to any stimulus, including light touch, and may be severely painful. Children with shingles usually have less severe symptoms than adults.

The blisters begin to dry and scab about 5 days after they appear. Until scabbing occurs, the blisters contain varicella-zoster virus, which can cause chickenpox if transmitted to susceptible people. Blisters that cover large areas of skin or persist for more than 2 weeks usually indicate that the immune system is not functioning properly.

One attack of shingles usually gives a person lifelong immunity from further attacks; fewer than 5% of people have further attacks. Scarring of the skin, which can be extensive, may occur, but most people recover without any lasting effects.

A few people, more commonly older people, continue to have chronic pain in the area (postherpetic neuralgia). Involvement of the part of the facial nerve leading to the eye can be quite serious, and if it is not treated properly, vision may be affected.

It may be difficult to diagnose shingles before the blisters appear, but the location of the initial pain in a vague band on one side of the body can be a useful clue. Depending on the nerves involved, the pain may resemble that caused by appendicitis, a kidney stone or gallstone, or inflammation of the large intestine. However, once the blisters appear in the typical pattern following a nerve root, the diagnosis is usually clear. Laboratory tests are seldom performed but may be used to confirm the diagnosis.

Polyoma (BK) virus infections are acquired early in childhood, and 60-80% of adults in the U.S. test seropositive for these viruses. The majority of infections are subclinical and lead to viral latency within the kidney. Reactivation occurs in transplant recipients as a result of immunosuppressive therapy. Serologic evidence suggests that the donor kidney may act as the vehicle of transmission. BK virus is shed in the urine of 10-60% of patients after renal transplantation, but clinically significant interstitial nephritis is infrequent.

The typical presentation is a rise in the serum creatinine, which cannot be distinguished from rejection or drug toxicity on clinical grounds. A few cases show hydronephrosis on ultrasound examination at the time of allograft biopsy. Intravenous pyelography in these patients may demonstrate a ureteric stricture. Hemorrhagic cystitis is described after bone marrow transplantation.

Needle biopsy of the allograft kidney shows a mixed interstitial inflammatory infiltrate with focal tubular injury. The tubular epithelium shows marked anisonucleosis, nuclear atypia, and basophilic or amphophilic intranuclear inclusions. Tubulitis is frequently present, and satisfies the Banff criteria for acute rejection. In addition biopsies may show clusters of neutrophils in the tubular lumen suggestive of pyelonephritis.

Polyoma virus inclusions in biopsy tissue are intranuclear with a homogenous basophilic or amphophilic appearance, which is quite distinct from cytomegalovirus, herpes virus and adenovirus inclusions. Definite identification may be done by immunohistochemistry, in-situ hybridization, or PCR. Electron microscopy is also a useful tool in the differential diagnosis. Polyoma virus particles measure 45-55 nm, while adenovirus measures 70-90 nm. Herpes simplex and cytomegalovirus are enveloped virions with a size range of 120-160 nm. In formalin fixed tissue viral particles can appear to be smaller than their expected size due to shrinkage artifact.

Both types of herpes simplex virus (HSV), HSV-1 and HSV-2, can cause oral or genital infection. Most often, HSV-1 causes oral infections such as gingivostomatitis, herpes labialis, and herpes keratitis. HSV-2 usually causes genital lesions. Transmission of HSV occurs from close contact with an individual who is actively shedding virus. Viral shedding generally occurs from lesions but can occur even when lesions are not apparent.

After the initial infection, HSV remains dormant in nerve ganglia from which it can periodically emerge, causing symptoms. Recurrent herpetic eruptions are precipitated by overexposure to sunlight, febrile illnesses, physical or emotional stress, immunosuppression, or unknown stimuli. Recurrent eruptions are generally less severe, and generally occur less frequently over time.

Mucocutaneous infection is the most common manifestation of herpes simplex virus disease. Lesions may appear anywhere on the skin or mucosa but are most frequent around or in the mouth or on the lips, conjunctiva and cornea, and genitals. Generally, after a prodromal period (typically <6 hours in recurrent HSV-1) of tingling discomfort or itching, clusters of small, tense vesicles appear on an erythematous base. Clusters vary in size from 0.5 to 1.5 cm but may coalesce. Lesions on the nose, ears, eyes, fingers, or genitals may be particularly painful. Vesicles typically persist for a few days, then dry, forming a thin, yellowish crust. Healing generally occurs 8 to 12 days after onset. Lesions usually heal completely, but recurrent lesions at the same site may cause atrophy and scarring. Skin lesions can develop secondary bacterial infection. In subjects with depressed cell-mediated immunity from HIV infection or other causes, prolonged or progressive lesions may persist for weeks or longer. Localized infections can disseminate, particularly, and often dramatically, in immunocompromised subjects.

Acute herpetic gingivostomatitis usually results from primary infection with HSV-1, typically in children. Occasionally, through oral-genital contact, the cause is HSV-2. Intraoral and gingival vesicles rupture, usually within several hours to 1 or 2 days, to form ulcers. Fever and pain often occur. Difficulty in eating and drinking may lead to dehydration. After resolution, the virus resides dormant in the semilunar ganglion.

Herpes labialis is usually a secondary outbreak of HSV. It develops as ulcers (cold sores) on the vermilion border of the lip or, much less commonly, as ulcerations of the mucosa of the hard palate.

Herpetic whitlow, a swollen, painful, erythematous lesion of the distal phalanx, results from inoculation of HSV through the skin and is most common in health care practitioners. Genital herpes is the most common ulcerative sexually transmitted disease in developed countries. It is usually caused by HSV-2, although 10 to 30% involve HSV-1. Primary lesions develop 4 to 7 days after contact. The vesicles usually erode to form ulcers that may coalesce. Lesions may occur on the prepuce, glans penis, and penile shaft in men and on the labia, clitoris, perineum, vagina, and cervix in women. The lesions may also occur around the anus and in the rectum. Genital HSV infection may cause urinary hesitancy, dysuria, urinary retention, or constipation. Severe sacral neuralgia may occur. Scarring may follow healing, and recurrences occur in 80% with HSV-2 and 50% with HSV-1. Primary genital lesions are usually more painful, prolonged, and widespread and are more likely to be bilateral and involve regional adenopathy and constitutional symptoms than recurrent genital lesions. Recurrent lesions may have severe prodromal symptoms and may involve the buttock, groin, or thigh.

Herpes simplex infection of the corneal epithelium (herpes simplex keratitis) produces pain, tearing, photophobia, and corneal ulcers that often have a branching pattern.

Neonatal herpes simplex infection develops in newborns, including in those whose mothers have no suggestion of current or past herpes infection. It is often transmitted during birth and usually involves HSV-2. It usually develops between the 1st and 4th week of life, often causing mucocutaneous vesicles or CNS involvement. It causes major morbidity and mortality.

Infection of the central nervous system (CNS) with herpes simplex, known as herpes encephalitis occurs sporadically and may be severe. Multiple early seizures are characteristic. Aseptic meningitis may result from HSV-2. It may involve lumbosacral myeloradiculitis which may produce urinary retention or obstipation.

Diagnosis of herpes simplex is often clinical based on characteristic lesions. Laboratory confirmation can be helpful, especially if infection is severe, the subject is immunocompromised or pregnant, or lesions are atypical. A Tzanck test (a superficial scraping from the base of a freshly ruptured vesicle stained with Wright's-Giemsa stain) often reveals multinucleate giant cells in HSV infection (or in varicella-zoster virus infection). Definitive diagnosis is with culture, seroconversion involving the appropriate serotype (in primary infections), and biopsy. Material for culture is obtained from a vesicle or the base of a freshly ulcerated lesion. HSV can sometimes be identified using direct immunofluorescence assay of scrapings of lesions. PCR of CSF and MRI are used to diagnose HSV encephalitis.

Measles is a highly contagious viral infection that produces various symptoms and a characteristic rash. The symptoms of measles begin about 7 to 14 days after infection. The infected subject (usually a child) first develops a fever, runny nose, sore throat, hacking cough, and red eyes. Sometimes, the eyes are sensitive to bright light. Tiny white spots (Koplik's spots) appear inside the mouth 2 to 4 days later. A mildly itchy rash appears 3 to 5 days after the start of symptoms. The rash begins in front of and below the ears and on the side of the neck as irregular, flat, red areas that soon become raised. The rash spreads within 1 to 2 days to the trunk, arms, and legs, as it begins to fade on the face. At the peak of the illness, the subject feels very sick, the rash is extensive, and the temperature may exceed 104° F. In 3 to 5 days, the temperature falls, the subject begins to feel better, and any remaining rash quickly fades.

The diagnosis is based on the typical symptoms and characteristic rash. Alternatively, laboratory diagnosis of measles can be done with confirmation of positive measles IgM antibodies or isolation of measles virus RNA from respiratory specimens.

Encephalitis occurs in about 1 of 1,000 children with measles. If encephalitis occurs, it often starts with a high fever, seizures, and coma, usually 2 days to 3 weeks after the rash appears. The illness may be brief, with recovery in about 1 week, or it may be prolonged, resulting in brain damage or death.

Secondary bacterial infections, such as pneumonia or an ear infection (otitis media), occur fairly often, and subjects with measles are especially susceptible to infection with streptococci bacteria.

Cytomegalovirus (CMV) is a type of herpes virus that generally causes disease mostly in infants infected before birth and in subjects who have a weakened immune system. Infection with CMV is very common. Blood tests show that 60 to 90% of adult humans have had a CMV infection at some time. Usually this infection produces no symptoms. Serious infections generally develop only in infants infected before birth and in subjects with an impaired immune system—for example, subjects with AIDS or those who have received an organ transplant. Subjects who have received an organ transplant are particularly susceptible to CMV infection because of the immunosuppressant drugs they receive as part of the transplantation process.

CMV spreads very easily. Infected subjects may shed the virus in their urine or saliva for months. The virus is also excreted in cervical mucus, semen, stool, and breast milk. Thus, both sexual and nonsexual transmission of the virus takes place. CMV infection may develop in subjects who receive infected blood.

CMV may cause symptoms soon after infection. It also has the ability to remain dormant in various tissues for the subject's lifetime. Various stimuli can cause the dormant CMV to become active and cause disease.

The vast majority of subjects infected with CMV have no symptoms. Occasionally, a healthy subject who is infected may feel ill and have a fever. CMV infection in adolescents and young adults can produce an illness with symptoms of fever and fatigue that resembles infectious mononucleosis. If a subject receives a transfusion of blood containing CMV, fever and sometimes liver inflammation may develop 2 to 4 weeks later.

A subject with a weakened immune system who becomes infected with CMV is particularly likely to develop a severe infection; serious illness and death may result. In subjects with AIDS, CMV infection is the most common viral complication. The virus tends to infect the retina of the eye (CMV retinitis), which can cause blindness. Infection of the brain (encephalitis) or ulcers of the intestine or esophagus may also develop.

In a pregnant woman, CMV infection can cause miscarriage, stillbirth, or death of the newborn. Death is caused by bleeding, anemia, or extensive damage to the liver or brain. Other disorders that may occur in the newborn include hearing loss and mental retardation.

CMV infection may develop gradually and not be recognized immediately. Healthcare professionals always consider the possibility of CMV infection in a subject with an impaired immune system. Once CMV infection is suspected, a healthcare professional conducts tests to detect the virus in body fluids or tissues. In newborns, the diagnosis is usually made by culturing the urine. In other subjects, healthcare professionals may be able to grow the virus from blood urine, throat swabs, bronchial lavages, lung specimens or tissue samples. Both qualitative and quantitative PCR testing for CMV are available as well, allowing physicians to monitor the viral load of CMV-infected patients.

Subjects who have been infected with CMV develop antibodies to the virus, and these antibodies persist in the body for the lifetime of that subject. A number of laboratory tests that detect these antibodies to CMV have been developed to determine if infection has occurred and are widely available from commercial laboratories.

For best diagnostic results, laboratory tests for CMV antibody should be performed by using paired serum samples. One blood sample should be taken upon suspicion of CMV, and another one taken within 2 weeks. A virus culture can be performed at any time the patient is symptomatic.

The enzyme-linked immunosorbent assay (or ELISA) is the most commonly available serologic test for measuring antibody to CMV. The result can be used to determine if acute infection, prior infection, or passively acquired maternal antibody in an infant is present. Other tests include various fluorescence assays, indirect hemagglutination, polymerase chain reaction (PCR) and latex agglutination.

In a subject with CMV retinitis, an eye doctor can see characteristic abnormalities in the retina.

The manifestations of human papillomavirus (HPV) infection depend on the location of the lesion and the type of virus. More than 100 different HPV types have been characterized. Some HPV types cause benign skin warts, or papillomas, for which the virus family is named. HPVs associated with the development of such “common warts” are transmitted environmentally or by casual skin-to-skin contact.

A group of about 30-40 HPVs are typically transmitted through sexual contact and infect the anogenital region. Some sexually transmitted HPVs, such as types 6 and 11, can cause genital warts. However, most HPV types that infect the genitals tend not to cause noticeable symptoms.

HPV-induced diseases include skin warts, genital warts, cancer and respiratory papillomatosis. Some “cutaneous” HPV types, such as HPV-1 and HPV-2, cause common skin warts. Common warts are often found on the hands and feet, but can also occur in other areas, such as the elbows or knees. Common warts have a characteristic cauliflower-like surface and are typically slightly raised above the surrounding skin. Cutaneous HPV types do not usually cause genital warts and are not associated with the development of cancer.

Genital or anal warts (condylomata acuminata or venereal warts) are the most easily recognized sign of genital HPV infection. Although a wide variety of HPV types can cause genital warts, types 6 and 11 account for about 90% of all cases. Most subjects who acquire genital wart-associated HPV types clear the infection rapidly without ever developing warts or any other symptoms. Subjects may transmit the virus to others even if they don't display overt symptoms of infections. HPV types that tend to cause genital warts are not the same ones that cause cervical cancer. However, since a subject can be infected with multiple types of HPV, the presence of warts does not rule out the possibility of high risk types of the virus also being present.

Several HPV types (including types 16, 18, 31 and 45) are also called “high risk” because they can lead to cervical cancer, as well as anal cancer, vulvar cancer, head and neck cancers, and penile cancer. HPV-induced cancers often have viral sequences integrated into the cellular DNA. Some of the HPV “early” genes, such as E6 and E7, are known to act as oncogenes that promote tumor growth and malignant transformation. A history of infection with one or more high-risk HPV types is believed to be a prerequisite for the development of cervical cancer. Sexually transmitted HPVs also cause a major fraction of anal cancers and approximately 25% of cancers of the mouth and upper throat (the oropharynx).

Most warts that are visible to the naked eye can be diagnosed correctly by history and physical examination alone. The use of a colposcope helps in assessing vaginal and cervical lesions and is helpful in the diagnosis of oral and cutaneous HPV disease as well. Pap smears prepared from cervical scrapings often show cytologic evidence of HPV infection. Persistent or atypical lesions are biopsied and examined by routine histologic methods. The most sensitive and specific methods of virologic diagosis entail the use of techniques such as such as polymerase chain reaction (PCR) or the hybrid capture assay to detect HPV nucleic acids and to identify specific virus types.

The compounds of the invention are administered in effective amounts. An effective amount is a dosage of the therapeutic agent sufficient to provide a medically desirable result. An effective amount means that amount necessary to delay the onset of, inhibit the progression of or halt altogether the onset or progression of the particular condition or disease being treated. In the treatment of viral diseases, for example, in general, an effective amount will be that amount necessary to inhibit viral replication, reduce viral load, or reduce one or more signs or symptoms of the disease. When administered to a subject, effective amounts will depend, of course, on the particular condition being treated; the severity of the condition; individual patient parameters including age, physical condition, size and weight, concurrent treatment, frequency of treatment, and the mode of administration. These factors are well known to those of ordinary skill in the art and can be addressed with no more than routine experimentation. In some embodiments, it is preferred to use a maximum dose, that is, the highest safe dose according to sound medical judgment.

An effective amount typically will vary from about 0.001 mg/kg to about 1000 mg/kg, from about 0.01 mg/kg to about 750 mg/kg, from about 0.1 mg/kg to about 500 mg/kg, from about 1.0 mg/kg to about 250 mg/kg, from about 10.0 mg/kg to about 150 mg/kg in one or more dose administrations daily, for one or several days (depending of course of the mode of administration and the factors discussed above). Other suitable dose ranges include 1 mg to 10000 mg per day, 100 mg to 10000 mg per day, 500 mg to 10000 mg per day, and 500 mg to 1000 mg per day. In some particular embodiments, the amount is less than 10,000 mg per day with a range of 750 mg to 9000 mg per day.

Actual dosage levels of active ingredients in the pharmaceutical compositions of the invention can be varied to obtain an amount of the active compound(s) that is effective to achieve the desired therapeutic response for a particular patient, compositions, and mode of administration. The selected dosage level depends upon the activity of the particular compound, the route of administration, the severity of the condition being treated, the condition, and prior medical history of the patient being treated. However, it is within the skill of the art to start doses of the compound at levels lower than required to achieve the desired therapeutic effort and to gradually increase the dosage until the desired effect is achieved.

The compounds and pharmaceutical compositions of the invention can be administered to a subject by any suitable route. For example, the compositions can be administered orally, including sublingually, rectally, parenterally, intracistemally, intravaginally, intraperitoneally, topically and transdermally (as by powders, ointments, or drops), bucally, or nasally. The term “parenteral” administration as used herein refers to modes of administration other than through the gastrointestinal tract, which include intravenous, intramuscular, intraperitoneal, intrasternal, intramammary, intraocular, retrobulbar, intrapulmonary, intrathecal, subcutaneous and intraarticular injection and infusion. Surgical implantation also is contemplated, including, for example, embedding a composition of the invention in the body such as, for example, in the brain, in the abdominal cavity, under the splenic capsule, brain, or in the cornea.

Compounds of the present invention also can be administered in the form of liposomes. As is known in the art, liposomes generally are derived from phospholipids or other lipid substances. Liposomes are formed by mono- or multi-lamellar hydrated liquid crystals that are dispersed in an aqueous medium. Any nontoxic, physiologically acceptable, and metabolizable lipid capable of forming liposomes can be used. The present compositions in liposome form can contain, in addition to a compound of the present invention, stabilizers, preservatives, excipients, and the like. The preferred lipids are the phospholipids and the phosphatidyl cholines (lecithins), both natural and synthetic. Methods to form liposomes are known in the art. See, for example, Prescott, Ed., Methods in Cell Biology, Volume XIV, Academic Press, New York, N.Y. (1976), p. 33, et seq.

Dosage forms for topical administration of a compound of this invention include powders, sprays, ointments, and inhalants as described herein. The active compound is mixed under sterile conditions with a pharmaceutically acceptable carrier and any needed preservatives, buffers, or propellants which may be required. Ophthalmic formulations, eye ointments, powders, and solutions also are contemplated as being within the scope of this invention.

Pharmaceutical compositions of the invention for parenteral injection comprise pharmaceutically acceptable sterile aqueous or nonaqueous solutions, dispersions, suspensions, or emulsions, as well as sterile powders for reconstitution into sterile injectable solutions or dispersions just prior to use. Examples of suitable aqueous and nonaqueous carriers, diluents, solvents, or vehicles include water ethanol, polyols (such as, glycerol, propylene glycol, polyethylene glycol, and the like), and suitable mixtures thereof, vegetable oils (such, as olive oil), and injectable organic esters such as ethyl oleate. Proper fluidity can be maintained, for example, by the use of coating materials such as lecithin, by the maintenance of the required particle size in the case of dispersions, and by the use of surfactants.

These compositions also can contain adjuvants such as preservatives, wetting agents, emulsifying agents, and dispersing agents. Prevention of the action of microorganisms can be ensured by the inclusion of various antibacterial and antifungal agents, for example, paraben, chlorobutanol, phenol sorbic acid, and the like. It also may be desirable to include isotonic agents such as sugars, sodium chloride, and the like. Prolonged absorption of the injectable pharmaceutical form can be brought about by the inclusion of agents which delay absorption, such as aluminum monostearate and gelatin.

In some cases, in order to prolong the effect of the drug, it is desirable to slow the absorption of the drug from subcutaneous or intramuscular injection. This result can be accomplished by the use of a liquid suspension of crystalline or amorphous materials with poor water solubility. The rate of absorption of the drug then depends upon its rate of dissolution which, in turn, may depend upon crystal size and crystalline form. Alternatively, delayed absorption of a parenterally administered drug from is accomplished by dissolving or suspending the drug in an oil vehicle.

Injectable depot forms are made by forming microencapsule matrices of the drug in biodegradable polymers such a polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations also are prepared by entrapping the drug in liposomes or microemulsions which are compatible with body tissue.

The injectable formulations can be sterilized, for example, by filtration through a bacterial- or viral-retaining filter, or by incorporating sterilizing agents in the form of sterile solid compositions which can be dissolved or dispersed in sterile water or other sterile injectable medium just prior to use.

The invention provides methods for oral administration of a pharmaceutical composition of the invention. Oral solid dosage forms are described generally in Remington's Pharmaceutical Sciences, 18th Ed., 1990 (Mack Publishing Co. Easton Pa. 18042) at Chapter 89. Solid dosage forms for oral administration include capsules, tablets, pills, powders, troches or lozenges, cachets, pellets, and granules. Also, liposomal or proteinoid encapsulation can be used to formulate the present compositions (as, for example, proteinoid microspheres reported in U.S. Pat. No. 4,925,673). Liposomal encapsulation may include liposomes that are derivatized with various polymers (e.g., U.S. Pat. No. 5,013,556). In general, the formulation includes a compound of the invention and inert ingredients which protect against degradation in the stomach and which permit release of the biologically active material in the intestine.

In such solid dosage forms, the active compound is mixed with, or chemically modified to include, a least one inert, pharmaceutically acceptable excipient or carrier. The excipient or carrier preferably permits (a) inhibition of proteolysis, and (b) uptake into the blood stream from the stomach or intestine. In a most preferred embodiment, the excipient or carrier increases uptake of the compound, overall stability of the compound and/or circulation time of the compound in the body. Excipients and carriers include, for example, sodium citrate or dicalcium phosphate and/or (a) fillers or extenders such as starches, lactose, sucrose, glucose, cellulose, modified dextrans, mannitol, and silicic acid, as well as inorganic salts such as calcium triphosphate, magnesium carbonate and sodium chloride, and commercially available diluents such as FAST-FLO®, EMDEX®, STA-RX 1500®, EMCOMPRESS® and AVICEL®, (b) binders such as, for example, methylcellulose ethylcellulose, hydroxypropylmethyl cellulose, carboxymethylcellulose, gums (e.g., alginates, acacia), gelatin, polyvinylpyrrolidone, and sucrose, (c) humectants, such as glycerol, (d) disintegrating agents, such as agar-agar, calcium carbonate, potato or tapioca starch, alginic acid, certain silicates, sodium carbonate, starch including the commercial disintegrant based on starch, EXPLOTAB®, sodium starch glycolate, AMBERLITE®, sodium carboxymethylcellulose, ultramylopectin, gelatin, orange peel, carboxymethyl cellulose, natural sponge, bentonite, insoluble cationic exchange resins, and powdered gums such as agar, karaya or tragacanth; (e) solution retarding agents such a paraffin, (f) absorption accelerators, such as quaternary ammonium compounds and fatty acids including oleic acid, linoleic acid, and linolenic acid (g) wetting agents, such as, for example, cetyl alcohol and glycerol monosterate, anionic detergent surfactants including sodium lauryl sulfate, dioctyl sodium sulfosuccinate, and dioctyl sodium sulfonate, cationic detergents, such as benzalkonium chloride or benzethonium chloride, nonionic detergents including lauromacrogol 400, polyoxyl 40 stearate, polyoxyethylene hydrogenated castor oil 10, 50 and 60, glycerol monostearate, polysorbate 40, 60, 65, and 80, sucrose fatty acid ester, methyl cellulose and carboxymethyl cellulose; (h) absorbents, such as kaolin and bentonite clay, (i) lubricants, such as talc, calcium sterate, magnesium stearate, solid polyethylene glycols, sodium lauryl sulfate, polytetrafluoroethylene (PTFE), liquid paraffin, vegetable oils, waxes, CARBOWAX® 4000, CARBOWAX® 6000, magnesium lauryl sulfate, and mixtures thereof; (j) glidants that improve the flow properties of the drug during formulation and aid rearrangement during compression that include starch, talc, pyrogenic silica, and hydrated silicoaluminate. In the case of capsules, tablets, and pills, the dosage form also can comprise buffering agents.

Solid compositions of a similar type also can be employed as fillers in soft and hard-filled gelatin capsules, using such excipients as lactose or milk sugar, as well as high molecular weight polyethylene glycols and the like.

The solid dosage forms of tablets, dragees, capsules, pills, and granules can be prepared with coatings and shells, such as enteric coatings and other coatings well known in the pharmaceutical formulating art. They optionally can contain opacifying agents and also can be of a composition that they release the active ingredients(s) only, or preferentially, in a part of the intestinal tract, optionally, in a delayed manner. Exemplary materials include polymers having pH sensitive solubility, such as the materials available as EUDRAGIT® Examples of embedding compositions which can be used include polymeric substances and waxes.

The active compounds also can be in micro-encapsulated form, if appropriate, with one or more of the above-mentioned excipients.

Liquid dosage forms for oral administration include pharmaceutically acceptable emulsions, solutions, suspensions, syrups, and elixirs. In addition to the active compounds, the liquid dosage forms can contain inert diluents commonly used in the art, such as, for example, water or other solvents, solubilizing agents and emulsifiers, such as ethyl alcohol, isopropyl alcohol ethyl carbonate ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydroflirfuryl alcohol, polyethylene glycols, fatty acid esters of sorbitan, and mixtures thereof.

Besides inert diluents, the oral compositions also can include adjuvants, such as wetting agents, emulsifying and suspending agents, sweetening, coloring, flavoring, and perfuming agents. Oral compositions can be formulated and further contain an edible product, such as a beverage.

Suspensions, in addition to the active compounds, can contain suspending agents such as, for example ethoxylated isostearyl alcohols, polyoxyethylene sorbitol and sorbitan esters, microcrystalline cellulose, aluminum metahydroxide, bentonite, agar-agar, tragacanth, and mixtures thereof.

Also contemplated herein is pulmonary delivery of the compounds of the invention. The compound is delivered to the lungs of a mammal while inhaling, thereby promoting the traversal of the lung epithelial lining to the blood stream. See, Adjei et al., Pharmaceutical Research 7:565-569 (1990); Adjei et al., International Journal of Pharmaceutics 63:135-144 (1990) (leuprolide acetate); Braquet et al., Journal of Cardiovascular Pharmacology 13 (supp1.5): s. 143-146 (1989)(endothelin-1); Hubbard et al., Annals of Internal Medicine 3:206-212 (1989)(α1-antitrypsin); Smith et al., J. Clin. Invest. 84:1145-1146 (1989) (α1-proteinase); Oswein et al., “Aerosolization of Proteins,” Proceedings of Symposium on Respiratory Drug Delivery II, Keystone, Colo., March, 1990 (recombinant human growth hormone); Debs et al., The Journal of Immunology 140:3482-3488 (1988) (interferon-γ and tumor necrosis factor α) and Platz et al., U.S. Pat. No. 5,284,656 (granulocyte colony stimulating factor).

Contemplated for use in the practice of this invention are a wide range of mechanical devices designed for pulmonary delivery of therapeutic products, including, but not limited to, nebulizers, metered dose inhalers, and powder inhalers, all of which are familiar to those skilled in the art.

Some specific examples of commercially available devices suitable for the practice of the invention are the ULTRAVENT® nebulizer, manufactured by Mallinckrodt, Inc., St. Louis, Mo.; the ACORN II® nebulizer, manufactured by Marquest Medical Products, Englewood, Colo.; the VENTOL® metered dose inhaler, manufactured by Glaxo Inc., Research Triangle Park, N.C.; and the SPINHALER® powder inhaler, manufactured by Fisons Corp., Bedford, Mass.

All such devices require the use of formulations suitable for the dispensing of a compound of the invention. Typically, each formulation is specific to the type of device employed and can involve the use of an appropriate propellant material, in addition to diluents, adjuvants, and/or carriers useful in therapy.

The composition is prepared in particulate form, preferably with an average particle size of less than 10 μm, and most preferably 0.5 to 5 μm, for most effective delivery to the distal lung.

Carriers include carbohydrates such as trehalose, mannitol, xylitol, sucrose, lactose, and sorbitol. Other ingredients for use in formulations may include lipids, such as DPPC, DOPE, DSPC and DOPC, natural or synthetic surfactants, polyethylene glycol (even apart from its use in derivatizing the inhibitor itself), dextrans, such as cyclodextran, bile salts, and other related enhancers, cellulose and cellulose derivatives, and amino acids.

Also, the use of liposomes, microcapsules or microspheres, inclusion complexes, or other types of carriers is contemplated.

Formulations suitable for use with a nebulizer, either jet or ultrasonic, typically comprise a compound of the invention dissolved in water at a concentration of about 0.1 to 25 mg of biologically active protein per mL of solution. The formulation also can include a buffer and a simple sugar (e.g., for protein stabilization and regulation of osmotic pressure). The nebulizer formulation also can contain a surfactant to reduce or prevent surface-induced aggregation of the inhibitor composition caused by atomization of the solution in forming the aerosol.

Formulations for use with a metered-dose inhaler device generally comprise a finely divided powder containing the inhibitor compound suspended in a propellant with the aid of a surfactant. The propellant can be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid also can be useful as a surfactant.

Formulations for dispensing from a powder inhaler device comprise a finely divided dry powder containing the inhibitor and also can include a bulking agent, such as lactose, sorbitol, sucrose, mannitol, trehalose, or xylitol, in amounts which facilitate dispersal of the powder from the device, e.g., 50 to 90% by weight of the formulation.

Nasal delivery of the compounds and composition of the invention also is contemplated. Nasal delivery allows the passage of the compound or composition to the blood stream directly after administering the therapeutic product to the nose, without the necessity for deposition of the product in the lung. Formulations for nasal delivery include those with dextran or cyclodextran. Delivery via transport across other mucous membranes also is contemplated.

Compositions for rectal or vaginal administration are preferably suppositories which can be prepared by mixing the compounds of the invention with suitable nonirritating excipients or carriers, such as cocoa butter, polyethylene glycol, or suppository wax, which are solid at room temperature, but liquid at body temperature, and therefore melt in the rectum or vaginal cavity and release the active compound.

In order to facilitate delivery of compounds across cell and/or nuclear membranes, compositions of relatively high hybrophobicity are preferred. Compounds can be modified in a manner which increases hydrophobicity, or the compounds can be encapsulated in hydrophobic carriers or solutions which result in increased hydrophobicity.

Generally dosage levels of about 0.1 to about 1000 mg, about 0.5 to about 500 mg, about 1 to about 250 mg, about 1.5 to about 100, and preferably of about 5 to about 20 mg of active compound per kilogram of body weight per day are administered orally or intravenously. If desired, the effective daily dose can be divided into multiple doses for purposes of administration, e.g., two to four separate doses per day.

The invention also encompasses methods of synthesizing PMEA and methods of conjugating oleic acid or oleyl alcohol and PMEA. Synthetic processes are described herein, although one of skill in the art will recognize that there may be other possible synthetic methods.

In the preparation of the compositions, oleic acid or oleyl alcohol may be reacted with PMEA to produce conjugates. As used herein, “reacting” oleic acid or oleyl alcohol with PMEA means the oleic acid or oleyl alcohol and PMEA are contacted under appropriate conditions for a time sufficient to result in the covalent conjugation of PMEA and oleic acid or oleyl alcohol. Such conditions encompass standard chemistry methods, which may be determined by one of skill in the art.

The invention is exemplified by the following Examples and is illustrated herein in reference to treatment of certain types of experimentally defined viral diseases. In these illustrative treatments, standard state-of-the-art in vitro and in vivo models have been used.

### EXAMPLES

### Example 1

### Synthesis of PMEA (9-[2-(phosphonomethoxy)ethyl]adenine)diethyl Ester

PMEA diethyl ester was synthesized using the commercially available 2-chloroethyl chloromethyl ether as the starting material (Scheme 1).

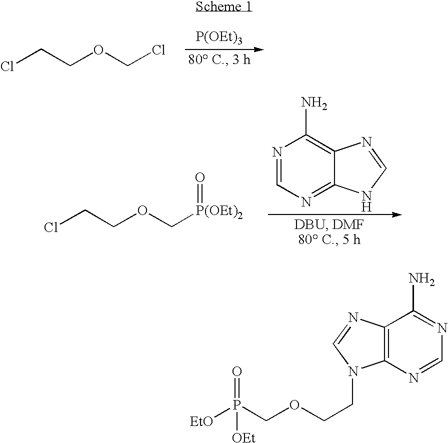

In a 25-mL, round-bottom flask adapted with a condenser and an addition funnel, triethyl phosphite (6.63 g, 40 mmol) was heated to 80° C. 2-chloroethyl chloromethyl ether (5.26 g, 40 mmol) was added drop wise and the reaction mixture was stirred at 80° C. for 3 h. The progress of the reaction was monitored by ¹H NMR spectroscopy. This solution was then added to an 80° C. suspension of adenine (5.41 g, 40 mmol) and DBU (6.09 g, 40 mmol) in DMF (80 mL). The reaction mixture was stirred at 80° C. for 5 h. The progress of the reaction was monitored by HPLC (method A). The reaction was incomplete, but did not go further. The solvent was then removed by rotary evaporation with concomitant addition of chloroform and cyclohexane to effect an azeotropic removal of the DMF. The product was purified by column chromatography (silica gel, chloroform to 8:1 chloroform/methanol). PMEA diethyl ester was obtained as a white powder (4.3 g, 18 mmol, 32%).

ESI MS: m/z 330 [M+H]⁺.

¹H NMR (300 MHz, DMSO-d₆): δ 8.2 (s, 1H, H₂), 8.1 (s, 1H, H₈), 7.2 (br s, 2H, NH₂), 4.3 (t, 2H, CH₂N), 3.9 (m, 8H, CH₂O, CH₂P), 1.2 (t, 6H, CH₃).

¹³C NMR (75 MHz, DMSO-d₆): δ 155.3, 152.7, 149.9, 141.4, 140.8, 119.0, 70.6, 65.1, 62.0, 42.7, 16.5.

### Synthesis of PMEA—Diethyl Ester Conjugates:

A. PMEA-Oleyl Amide: PMEA oleyl amide was synthesized using the CDI activated fatty acid. Oleyl amide was synthesized following the general procedure.

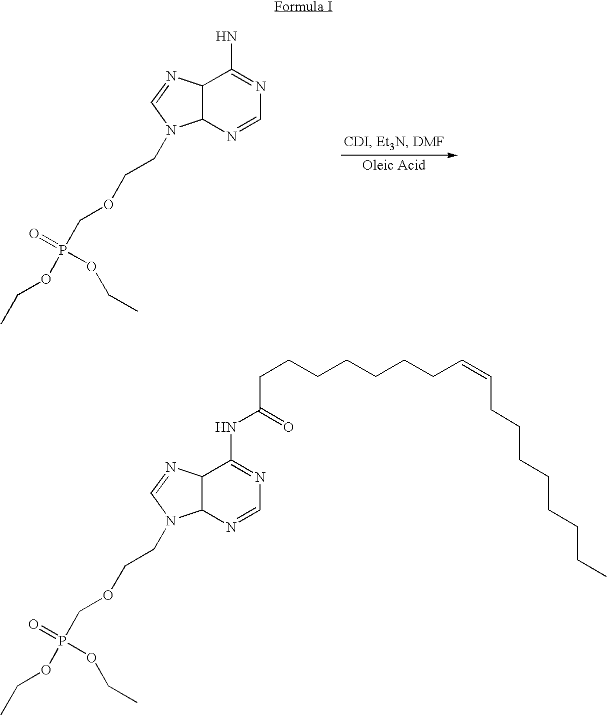

To a suspension of CDI (1.73 g, 10.6 mmol) in DMF (7.5 mL) under argon at 0° C., oleic acid (10.6 mmol) was added drop wise as a solution in DMF (4 mL). The clear solution resulting after the addition was stirred at 0° C. for 20 min and then at room temperature for 1 h. In a separate flask, triethylamine (1.07 g, 10.6 mmol) was added to a solution of PMEA diethyl ester 3 (700 mg, 2.1 mmol) in dry DMF (4 mL) and stirred for 10 min. To this solution was added drop wise the CDI activated ester at room temperature. After the addition, the reaction mixture was heated at 75° C. for 2.5 d. DMF was removed under high vacuum and the crude reaction mixture was taken up in ethyl acetate, washed with satd. NH₄Cl (2×15 mL) followed by satd. NaHCO₃ (2×15 mL), water (15 mL) and brine (15 mL). The combined organic layer was dried (Na₂SO₄) and concentrated at reduced pressure. The residue was purified by ISCO combiflash system using a gradient elution: 10% methanol in ethyl acetate to 50% methanol in ethyl acetate to afford the diethyl phosphonate (Formula I). Yield: 79-85%.

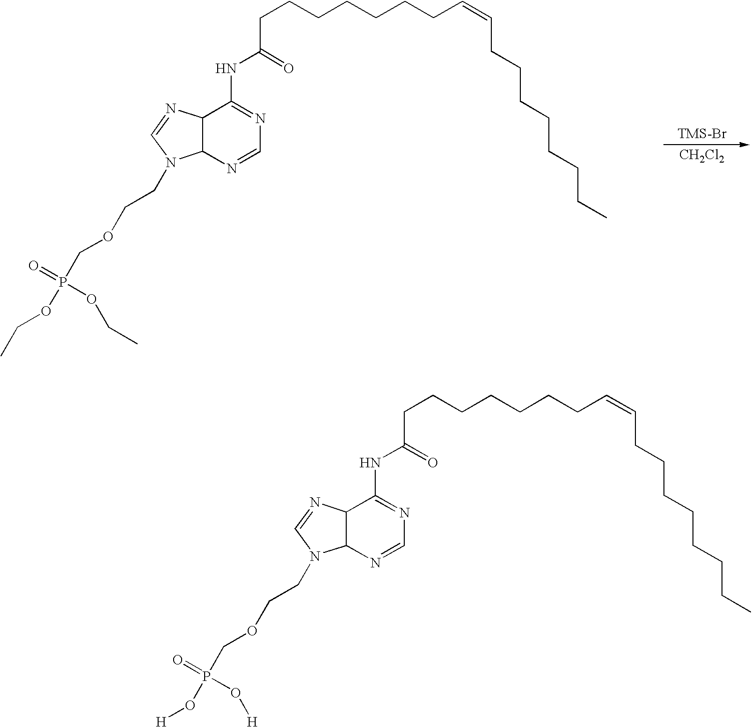

To a cold solution (0° C.) of the diethyl phosphonate (Formula I) (415 mg, 0.7 mmol) in anhydrous CH₂Cl₂ (6 mL) was added TMS-Br (859 mg, 5.6 mmol). The ice bath was removed and the reaction mixture was stirred at room temperature for 3 h. Solvent was then removed under reduced pressure. The residue was co-evaporated with CH₃CN (10 mL) to remove unreacted TMS-Br and ethyl bromide to afford the crude acid (Formula II). Yield: 84-90%.

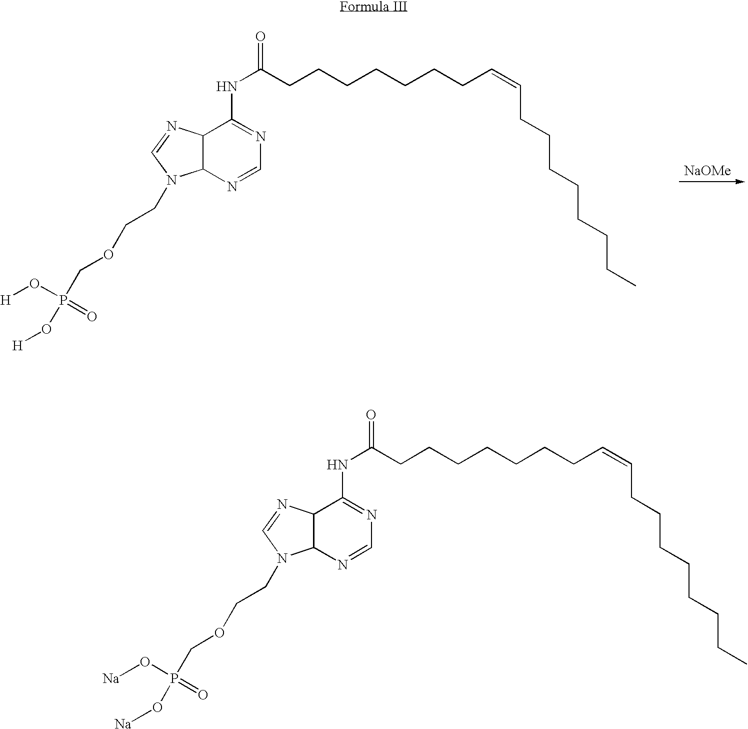

To a solution of the crude acid (Formula II) (119 mg, 0.22 mmol) in a mixture (1:1) of CHCl₃/MeOH (10 mL), NaOMe (96 mg, 4.4 mmol, 25 wt % in MeOH) was added and the solution was stirred for 2 h at 0° C. Solvent was removed under reduced pressure and the crude salt was dried at high vacuum overnight. The crude salt was washed with hexane (2×10 mL), ethyl acetate (2×10 mL) followed by EtOH (2×10 mL) to remove the impurities and dried under high vacuum to afford the disodium salt (Formula III). Yield: 60-62%.

At this point if the washings did not remove the impurities, a reverse phase column (C18) using H₂O:CH₃CN (70:30) as mobile phase was used for further purification.

B. PMEA Oleyl Carbamate: The carbamate was synthesized via the activation of the adenine with CDI followed by the addition of oleyl alcohol. Specifically, PMEA oleyl carbamate was synthesized following the procedure using oleyl alcohol.

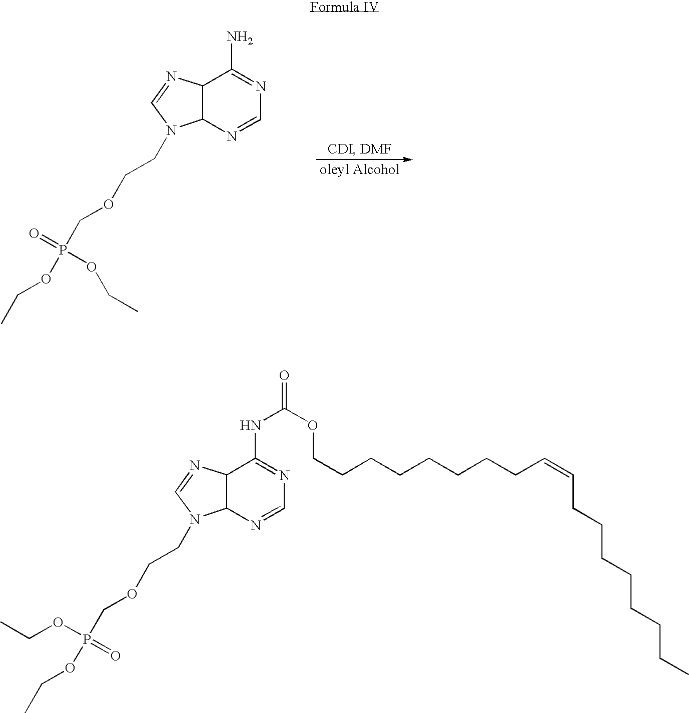

To a suspension of PMEA diethyl ester (500 mg, 1.52 mmol) in dry DMF (3 mL), CDI (369 mg, 2.28 mmol) was added and the reaction mixture was stirred at 105° C. for 2 h. The reaction mixture was cooled to 95° C. and oleyl alcohol (ROH) (2.28 mmol) was added neat. The oil bath was removed and the reaction mixture was stirred at room temperature overnight. DMF was removed at high vacuum and the crude reaction mixture was taken up in ethyl acetate, washed with satd. NH₄Cl (2×15 mL) followed by satd. NaHCO₃ (2×15 mL), water (15 mL) and brine (15 mL). The combined organic layer was dried (Na₂SO₄) and concentrated under reduced pressure. The residue was purified by ISCO combiflash system using a gradient elution: 10% methanol in ethyl acetate to 50% methanol in ethyl acetate to afford the diethyl phosphonate (Formula IV). Yield: 59-61%.

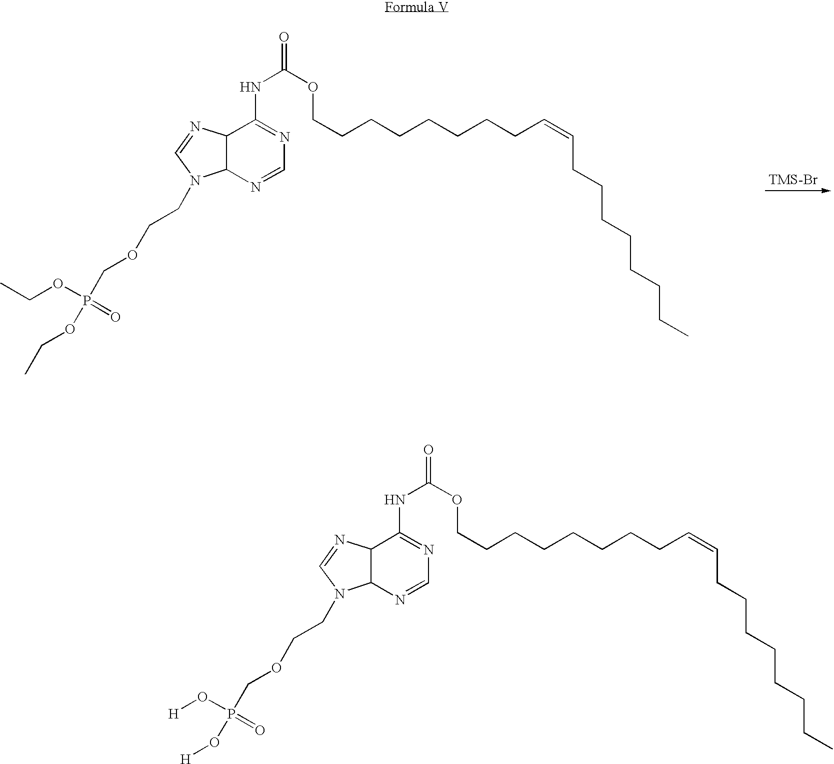

To a solution of diethyl phosphonate (Formula IV) (401 mg, 0.65 mmol) in dry CH₂Cl₂ (10 mL), TMS-Br (910 mg, 4.52 mmol) was added and the reaction mixture was stirred at room temperature for 3 h. Solvent was removed under reduced pressure and the residue was co-evaporated with CH₃CN (10 mL) to remove unreacted TMS-Br and ethyl bromide to afford the crude acid (Formula V). Yield: 89-95%.

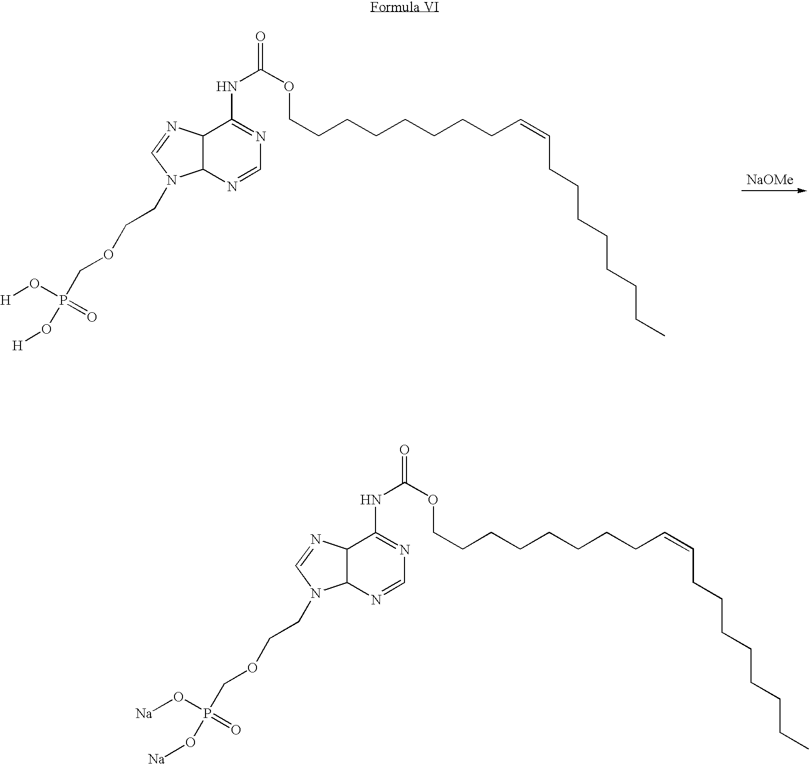

To a cold (−5° C.) solution of the crude acid (Formula V) (351 mg, 0.62 mmol) in a mixture (1:1) of CHCl₃/MeOH (24 mL), NaOMe (308 mg, 1.43 mmol, 25 wt % in MeOH) was added and the solution stirred for 1.5 h at −5° C. Solvent was removed under reduced pressure and the crude salt was dried at high vacuum. The crude salt was washed with hexane (2×10 mL), ethyl acetate (2×10 mL) followed by EtOH (2×10 mL) to remove the impurities and dried under high vacuum to afford Formula VI. Yield: 66%.

### Example 2

### Background

PMEA is an acyclic nucleoside phosphonate. Compound of Formula III and compound of Formula VI are lipid conjugates of PMEA, in which the lipid in compound of Formula III is oleic acid and the lipid in compound of Formula VI is oleyl alcohol. The assumption among those skilled in the art has always been that, like other fatty acid conjugates, the antiviral conjugates must be converted back to the parent compound to become activated to an effective antiviral agent. Therefore, the ultimate antiviral mechanism of action of the conjugates has until now been thought to be similar to that of the parent compound, PMEA. The fatty acids conjugated to PMEA are known to add favorable modulations of pharmacokinetics, pharmacodynamics, and tissue targeting. Once at or near the tissue target, the data seemed to indicate that the conjugates would be cleaved to the parent compound and inhibit viral replication by the parent's known mechanism.

### Summary of Data:

In vivo data: Compound of Formula III and compound of Formula VI have been tested against a mouse model of Hepatitis B virus in which the virus is carried on a promoter integrated into the mouse genome. Compounds with anti-hepatitis B activity decrease the amount of viral DNA in the circulation and the amount produced in the liver. The conjugates, compound of Formula III and compound of Formula VI, as well as unconjugated PMEA, were active against Hepatitis B in that model. However, the conjugates had a log or more anti Hepatitis B activity than did PMEA after i.p. administration.

In vitro data: PMEA is highly active in vitro against Hepatitis B. However, neither compound of Formula III nor compound of Formula VI has any measurable activity against Hepatitis B in vitro. These findings support the idea that the fatty acid conjugates must be first hydrolyzed back to the parent drug, PMEA, in order to have anti-Hepatitis B activity and that such metabolism does not occur in vitro.

Compound of Formula III and compound of Formula VI were tested against a number of other viruses as detailed in Table 1 below:

TABLE 1

| | | SI for Formula | SI for Formula |
|---|---|---|---|
| Virus | SI for PMEA | III compound | VI compound |
| HBV | 29 | 1 | 1 |
| HSV-1 (1) | ND | >17.8 | 1 |
| HSV-1 (2) | 1 | >143 | 1 |
| HSV-2 (1) | | >8.5 | 1 |
| HSV-2 (2) | 1 | 5.7 | 1 |
| HCMV | 1 | 1 | 444 |
| VZV (1) | ND | 1 | >3125 |
| VZV (2) | 1 | >435 | >213 |
| BK (polyoma) | | >213 | >22.5 |
| Cowpox (1) | | 1 | 1 |
| Cowpox (2) | | 1 | >1.1 |
| Vaccinia (1) | | 1 | 1 |
| Vaccinia (2) | | >2 | >1.5 |
| Measles (NR assay) | | >20 | 4 |
| Dengue | | 1 | 1 |
| Adeno | | 1 | 1 |
| Yellow Fever | | 1 | 1 |
| Punta Toro | | 1 | 1 |
| Flu A | | 1 | 1 |
| Flu B | | 1 | 1 |
| SARS | | 1 | 1 |
| Tacaribe | | 2 | 1 |
| PIV | | 1 | 1 |
| RSV A | | 1 | 1 |
| RV 2 | | 1 | 1 |
| VEE | | 1 | 1 |
| Rift Valley Fever | | 1 | 1 |
| Pichinde | | 1 | 1 |
| HPV-11 | | 2.5 | 2 |
| HPV-11 | | 7.8 | 1 |
| HPV-11 | | 4.2 | 1 |
| HPV-11 | | >25 | |

(1) or (2): the first or second replicate. Selectivity index (SI) is representative of each test compound's antiviral activity and is expressed as a ratio which is the IC50 or IC90 divided by the EC50. Generally, an SI of 5 or greater is indicative of positive antiviral activity, although other factors, such as a low SI for the positive control, are also taken into consideration. An SI of 1.0 means that no antiviral activity was seen at the doses tested or that the cellular toxicity and antiviral efficacy are equal, implying there would be no therapeutic advantage in using the compound to treat diseases.

The expected results for fatty acid conjugates are shown by the HBV (Hepatitis B virus) data above in which the parent compound, PMEA, has potent activity against the virus in vitro, whereas the fatty acid conjugates, compound of Formula III and compound of Formula VI, lack such activity. These results are what those skilled in the art would expect to discover because previous fatty acid conjugates such as DHA-paclitaxel (Bradley et al., Clin. Cancer Res. 7(10): 3229-3238, 2001) were inactive as intact conjugates, and only had therapeutic activity (microtubule assembly) after metabolism back to the parent compound, paclitaxel.

The results with HSV-1, HSV-2, HCMV, and VZV are in contrast to these with HBV. They are the opposite of what someone skilled in the art would expect to find. In these examples, PMEA was inactive against those viruses, yet one or both of the conjugates were highly active against the viruses. The most likely explanation for these results is that either the conjugates themselves, a spontaneous breakdown product of a conjugate, or a metabolite of a conjugate leads to antiviral activity in the absence of activity by the parent. These findings indicate that the compositions of the lipid-PMEA conjugates either possess themselves, or cause to form, antiviral activity beyond that of the parent PMEA itself and are thus unexpected.

### In Vitro Assays Used to Determine Antiviral Activity

Screening assays for activity against different viruses: All the screening assay systems showed specific inhibition of a biologic function, i.e., cytopathic effect (CPE) in susceptible human cells. In the CPE-inhibition assay, drug was added 1 hr prior to infection so the assay system will have maximum sensitivity and detect inhibitors of early replication such as adsorption to cells or membrane penetration as well as later molecular events. To rule out non-specific inhibition of virus binding to cells all compounds that show reasonable activity in the CPE assay were confirmed using a classical plaque reduction assay in which the drug was added 1 hr after infection. This assay was the one to which all other assay systems were compared. In the case where a compound blocked attachment, it showed up as a positive in the CPE assay, but may be negative by plaque assay. In this case, the plaque assay was repeated with drug being added prior to viral infection. Using this approach compounds were identified that inhibit virus adsorption. These assay systems also can be manipulated by increasing the pretreatment time in order to demonstrate antiviral activity with oligodeoxynucleotides and/or peptides and by delaying addition of drug after infection, information regarding which step in the virus life cycle is inhibited (i.e. early vs. late functions) can be gained.

Efficacy: In all the assays, a minimum of six drug concentrations, ranging from 0.03 to 100 μg/ml, were tested. These data produced dose response curves. From these data, the dose that inhibits viral replication by 50% (effective concentration 50; EC₅₀) was calculated using the computer software program MacSynergy II.

Toxicity: The same drug concentrations used to determine efficacy were also used on uninfected cells in each assay to determine toxicity of each experimental compound. The drug concentration that is cytotoxic to cells as determined by their failure to take up a vital strain, neutral red, (either inhibitory concentration IC₅₀, or cytotoxic concentration 50; CC₅₀) was determined.

Since many antiviral drugs need to be given parenterally, it was important to determine the toxicity of new compounds on dividing cells. Cell proliferation assays using human foreskin fibroblast (HFF) cells are very sensitive for detecting drug toxicity in dividing cells and the drug concentration that inhibits cell growth by 50% (IC₅₀) was calculated as described above. HFF cells are the most sensitive and predictive of toxicity for bone marrow cells.

Assessment of drug activity: To determine if each compound had sufficient antiviral activity that exceeded its level of toxicity, a selectivity index (SI) was calculated according to IC₅₀ or CC₅₀/EC₅₀. This index, also referred to as a therapeutic index, was used to determine if a compound warrants further study. For these studies, a compound that has an SI of 5 or greater was considered active.

### Laboratory Procedures for Determining Antiviral Efficacy and Toxicity

Preparation of Compounds for In Vitro Testing: if a Compound was Water Soluble, it was dissolved in tissue culture media without serum at 1 mg/ml and diluted for use as indicated below in the description of the assay system. If the compound was not water soluble, then it was dissolved in DMSO at a concentration of 10 mg/ml and diluted for use in each assay.

### Screening and Confirmation Assays

Cytopathic Effect Inhibition Assay: Low passage (3-10) HFF cells were trypsinized, counted, and seeded into 96 well tissue culture plates at a cell concentration of 2.5×10⁴ cells in 0.1 ml of MEM supplemented with 10% FBS. The cells were then incubated for 24 hr at 37° C. in a 5% CO₂-95% air, 90% humidified atmosphere. The media was then removed and 100 μl of MEM containing 2% FBS was added to all but the first row. In the first row, 125 μl of media containing the experimental drug was added in triplicate wells. Media alone was added to both cell and virus control wells. The drug in the first row of wells was then diluted serially 1:5 throughout the remaining wells by transferring 25 μl using a Beckman Bio-Mek Liquid Handling Machine. The plates were then incubated for 60 min and 100 μl of an appropriate virus concentration added to each well, excluding cell control wells which received 100 μl of MEM. The plates were then incubated at 37° C. in a CO₂ incubator for from three days to 14 days for slower growing viruses. After the incubation period, media was aspirated and the cells stained with a 0.1% crystal violet in formalin solution for 4 hr. The stain was then removed and the plates rinsed using tap water until all excess stain was removed. The plates were allowed to dry for 24 h and the amount of CPE in each row determined using a BioTek Multiplate Autoreader. EC₅₀ and IC₅₀ values were determined by comparing drug treated and untreated cells using a computer program.

Plaque reduction assay using semi-solid overlay: Two days prior to use, HFF cells were trypsinized, counted, and plated into six well plates and incubated at 37° C. with 5% CO₂ and 90% humidity. On the date of assay, the drug was made up at twice the desired concentration in 2×MEM and then serially diluted 1:5 in 2×MEM to give six concentrations of drug. The drug concentrations utilized were usually 200 μg/ml down to 0.06 μg/ml. The virus to be used was diluted in MEM containing 10% FBS to a desired concentration which gave 20-30 plaques per well. The media was then aspirated from the wells and 0.2 ml of virus was added to each well in triplicate with 0.2 ml of media being added to drug toxicity wells. The plates were then incubated for 1 hr with shaking every fifteen min. After the incubation period, an equal amount of 1% agarose was added to an equal volume of each drug dilution. This gave final drug concentrations from 0.03 μg/ml to 100 μg/ml and a final agarose overlay concentration of 0.5%. The drug agarose mixture was applied to each well in 2 ml volume and the plates were incubated for three days, after which the cells were stained with a 1.5% solution of neutral red. At the end of the 4-6 hr incubation period, the stain was aspirated, and plaques counted using a stereomicroscope at 10× magnification.

Plaque reduction assays using liquid medium overlay: Some large or highly charged molecules that were active in the CPE-inhibition assay were inactive in the plaque assay because drugs fail to diffuse through the agarose overlay. The procedure for the liquid overlay plaque assay is similar to that using the agarose overlay. The procedure for adding the virus is the same as for the regular plaque assay. The drugs were made up at the desired concentrations in MEM with 2% FBS. For viral assays, an antibody preparation was diluted 1:500 and added to the media that the drug was diluted in to limit extracellular spread of virus through the media. At the end of the incubation period for each assay, 2 ml of a 6.0% neutral red solution was added to each well and incubated for six hr. The liquid was then aspirated off and plaques counted using a stereomicroscope.

### Screening and Confirmation Assays for Toxicity

Neutral red uptake assay—HFF Cells: Twenty-four hr prior to assay, HFF cells were plated into 96 well plates at a concentration of 2.5×10⁴ cells per well. After 24 hr, the media was aspirated and 125 μl of each drug concentration was added to the first row of wells and then diluted serially 1:5 using the automated Bio-Mek Liquid Handling System in a manner similar to that used in the CPE assay. The plates were then incubated in a CO₂ incubator at 37° C. for seven days. At this time the media/drug was aspirated and 200 μl/well of 0.01% neutral red in Phosphate Buffered Saline was added. This mixture was incubated in the CO₂ incubator for 1 hr. The drug was aspirated and the cells were washed with a Nunc Plate Washer. After removing the Phosphate Buffered Saline wash, 200 μl/well of 50% ETOH/1% glacial acetic acid (in H₂O) was added. The plates were rotated for 15 min and the optical densities were read at 550 nm on a plate reader. CC₅₀ values were calculated using a computer program.

Cell proliferation assay in HFF cells: Twenty-four hr prior to assay, HFF cells were seeded in 6-well plates at a concentration of 2.5×10⁴ cells per well in MEM containing 10% FBS. On the day of the assay, drugs were diluted serially in MEM containing 10% FBS at increments of 1:5 covering a range from 100 μg/ml to 0.03 μg/ml. For drugs that had to be solubilized in DMSO, control wells received MEM containing 1.0% DMSO. The media from the wells was then aspirated and 2 ml of each drug concentration was then added to each well. The cells were then incubated in a CO₂ incubator at 37° C. for 72 hr. At the end of this time, the media-drug solution was removed and the cells washed. One ml of 0.25% trypsin was added to each well and incubated until the cells start to come off of the plate. The cell-media mixture was then pipetted up and down vigorously to break up the cell suspension and 0.2 ml of the mixture was added to 9.8 ml of Isoton III and counted using a Coulter Counter. Each sample was counted 3 times with 2 replicate wells per sample.

This invention is not limited in its application to the details of construction and the arrangement of components set forth in the above description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

We claim:

1. A compound having a structure: 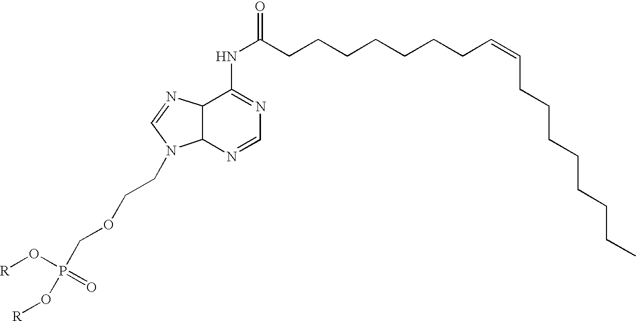
or a salt thereof,
wherein R represents —CH₂CH₃, hydrogen or a monovalent cation.

2. The compound of claim 1, wherein the monovalent cation is lithium, sodium or potassium.

3. The compound of claim 1 having a structure:

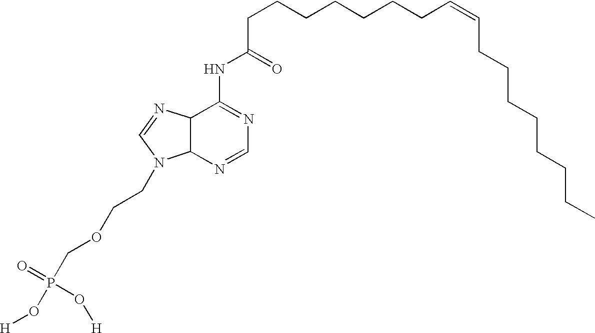

4. The compound of claim 1 having a structure:

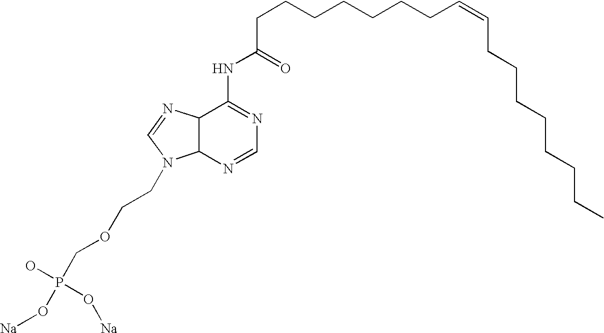

5. A pharmaceutical composition comprising the compound of claim 1 and a pharmaceutically acceptable carrier.

6. The pharmaceutical composition of claim 5 further comprising an agent other than the compound of claim 1.

7. The pharmaceutical composition of claim 6, wherein the agent is an antiviral agent.

8. The pharmaceutical composition of claim 7, wherein the antiviral agent is acyclovir, cidofovir famciclovir, foscarnet, gancyclovir, penciclovir, trifluridine, valacyclovir, valganciclovir, or vidarabine.

9. A method for treating a subject having a viral disease comprising:
administering to the subject an effective amount of a pharmaceutical composition of claim 1 to treat the viral disease, wherein the viral disease is caused by herpes simplex virus (HSV), varicella-zoster virus (VZV), measles virus, polyoma (BK) virus, or human papilloma virus.

10. The method of claim 9, wherein the herpes simplex virus is herpes simplex type I or herpes simplex type 2 virus.

11. The method of claim 10, wherein the herpes simplex virus type I disease is oral herpes infection.

12. The method of claim 10, wherein the herpes simplex virus type II disease is genital herpes infection.

13. The method of claim 9, wherein the varicella-zoster virus disease is chickenpox.

14. The method of claim 9, wherein the varicella-zoster virus disease is shingles.

15. A compound having a structure: 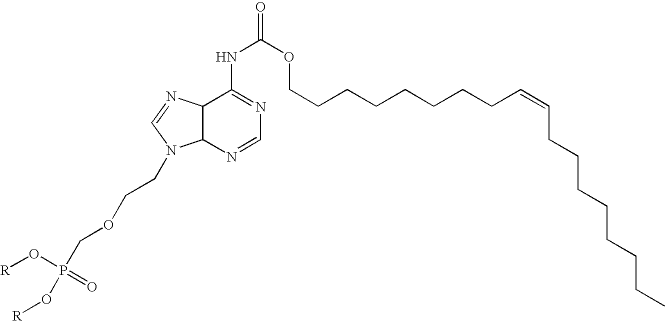
or a salt thereof,
wherein R represents —CH₂CH₃, hydrogen or a monovalent cation.

16. The compound of claim 15, wherein the monovalent cation is lithium, sodium or potassium.

17. The compound of claim 15 having a structure:

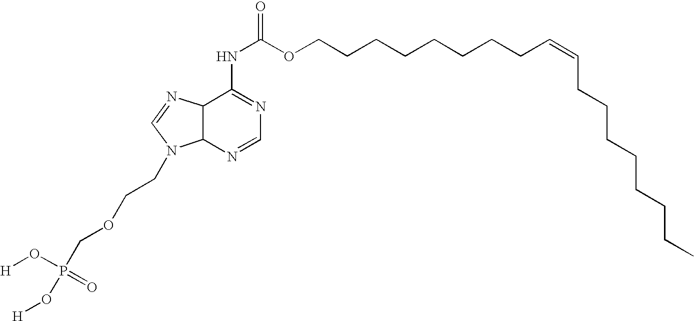

18. The compound of claim 15 having a structure:

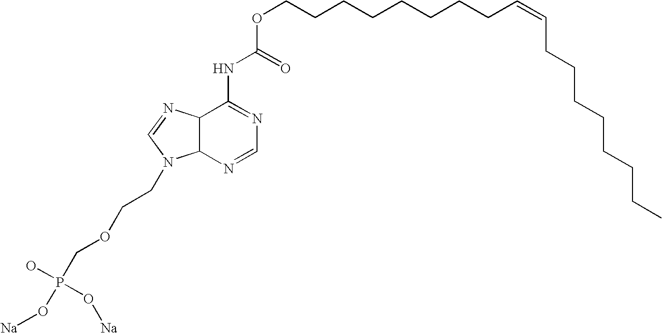

19. A pharmaceutical composition comprising the compound of claim 15 and a pharmaceutically acceptable carrier.

20. The pharmaceutical composition of claim 19 further comprising an agent other than a compound of claim 15.

21. The pharmaceutical composition of claim 20, wherein the agent is an antiviral agent.

22. The pharmaceutical composition of claim 21, wherein the antiviral agent is acyclovir, cidofovir famciclovir, foscarnet, gancyclovir, penciclovir, trifluridine, valacyclovir, valganciclovir, or vidarabine.

23. A method for treating a subject having a viral disease comprising:
administering to the subject an effective amount of a pharmaceutical composition of claim 15 to treat the viral disease, wherein the viral disease is caused by cytomegalovirus (CMV or HCMV), varicella-zoster virus (VZV), or polyoma (BK) virus.

24. The method of claim 23, wherein the varicella-zoster virus disease is chickenpox.

25. The method of claim 23, wherein the varicella-zoster virus disease is shingles.
